(19) United States
(12) Patent Application Publication
(10) Pub. No.: US 2025/0092118 A1
(43) Pub. Date: Mar. 20, 2025

(54) HIGHLY PURIFIED INSOLUBLE COLLAGEN FIBERS FOR MEDICAL APPLICATIONS

(71) Applicant: Shu-Tung and Alice Li Foundation Inc., Oakland, NJ (US)

(72) Inventors: Shu-Tung Li, Franklin Lakes, NJ (US); Solaiman Tarafder, Oakland, NJ (US)

(73) Assignee: Shu-Tung and Alice Li Foundation Inc., Oakland, NJ (US)

(21) Appl. No.: 18/829,752

(22) Filed: Sep. 10, 2024

Related U.S. Application Data
(60) Provisional application No. 63/582,686, filed on Sep. 14, 2023.

(51) Int. Cl. C07K 14/78; C07K 1/14

(52) U.S. Cl. CPC C07K 14/78; C07K 1/14

(57) ABSTRACT

This disclosure relates to development of biocompatible and resorbable type II collagen fibers for medical applications. Its characteristics and methods of preparation are also disclosed.

### CROSS-REFERENCE TO RELATED APPLICATIONS

This application claims the benefit under 35 U.S.C. § 119 (e) of the earlier filing date of U.S. Provisional Patent No. 63/582,686 filed on Sep. 14, 2023, which is hereby incorporated by reference in its entirety.

### FIELD OF THE INVENTION

This invention relates generally to highly purified insoluble collagen fibers for medical applications.

### BACKGROUND OF THE INVENTION

Type II collagen is the major extracellular matrix component in all cartilaginous tissues. In addition to type II collagen, adult articular cartilage contains a minor amount of type IX and type XI collagens that are physically and chemically associated with the type II collagen fibers. Therefore, type II collagen fibril is a heteropolymer with type IX collagen molecules covalently linked to the surface of type II collagen fiber and collagen XI, forming the filamentous template of the fibril. In addition, articular cartilage also contains about 10-15% dry weight of polysaccharide moieties in the form of glycosaminoglycans (GAGs) that are covalently linked to a protein core. The protein-GAG complex is named proteoglycan. Finally, numerous proteoglycans are covalently linked to a hyaluronic acid molecule, forming a giant complex molecule called aggrecan.

The strong physical and mechanical association between the two major classes of molecules creates a viscoelastic property that bestows the articular cartilage with an extraordinary mechanical characteristic of the tissue to function in the knee joint for weight bearing, weight distribution, knee movement, knee protection, and knee stability. Beneath the cartilage layer, there is a calcified cartilage region (tide mark) where chondrocytes are atrophied and begin to synthesize type X collagen, which plays a significant role in the endochondral ossification process.

Given the complex structure of the extracellular matrix of the articular cartilage, along with low cell content and avascular nature of the tissue, it is extremely challenging to treat the articular cartilage tissue once it is injured from either disease or sports. It is also exceedingly difficult to repair injured or diseased articular cartilage tissue via a tissue engineering approach to fabricate extracellular matrices (ECMs) that mimic the ECMs of the native tissues for guiding articular cartilage tissue repair and regeneration.

Unlike type I collagen fibers in tendons and ligaments where type I collagen fibers can be isolated and purified for tissue engineering applications, type II collagen molecules in cartilages are crosslinked in vivo, involving three collagen molecules. These crosslinks initiated from the endogenous enzymatic modification of hydroxylysines to aldehyde-containing side chains, which form stable pyridinoline-based crosslinks. During these in vivo fiber-genic processes, type IX and type XI collagens play significant roles in regulating fiber growth and fiber structure. As a result, the diameter of type II collagen fibers is small, generally in the nanometer (nm) range instead of in the micrometer range as seen in the type I collagen.

Despite the complex nature of the type II collagen fibers, it is still feasible to release type II collagen molecules from the fibers via pepsin digestion to cleave the intermolecular crosslinks at the telopeptide region of the molecule. However, purifying insoluble type II collagen fibers from native cartilage has not been feasible due to the complexity of the tissue, and the existing methods often led to partial degradation of the type II collagen molecules, resulting in significant reduction of the hydrothermal shrinkage temperature of the collagen fibers.

Thus, there remains a need for improved processes to obtain type II collagen fibers for medical applications.

### SUMMARY OF THE INVENTION

This disclosure provides purified insoluble type II collagen fibers. In some embodiments, the purified insoluble type II collagen fibers comprise (a) less than 0.6% (w/w) uronic acid, (b) less than 50 ng/mg of nucleic acid molecules, (c) from 12.5% (w/w) to 15.5% (w/w) of hydroxyproline, (d) more than 97% (w/w) of type II collagen, and (e) less than 3% (w/w) of collagens other than type II collagen and glycosaminoglycans (GAGs).

In some embodiments, the type II collagen fibers comprise less than 0.3% (w/w) of the uronic acid. In some embodiments, the type II collagen fibers comprise less than 0.1% (w/w) of the uronic acid.

In some embodiments, the type II collagen fibers comprise less than 30 ng/mg of the nucleic acid molecules. In some embodiments, the type II collagen fibers comprise less than 10 ng/mg of the nucleic acid molecules. In some embodiments, the nucleic acid molecules comprise DNA.

In some embodiments, the type II collagen fibers comprise more than 13% (w/w) of the hydroxyproline. In some embodiments, the type II collagen fibers comprise more than 14% (w/w) of the hydroxyproline. In some embodiments, the type II collagen fibers comprise more than 15% (w/w) of the hydroxyproline.

In some embodiments, the type II collagen fibers comprise less than 1% (w/w) of collagens other than type II collagen and GAGs. In some embodiments, the type II collagen fibers comprise less than 0.5% (w/w) of collagens other than type II collagen and GAGs.

In some embodiments, the collagen fibers are isolated from cartilage tissues of animals or humans.

In some embodiments, the tissues are knee joint articular cartilages. In some embodiments, the tissues are chicken sternum.

In some embodiments, the collagen fibers are isolated by genetic engineering technologies.

In some embodiments, the collagen fibers have stability as measured by a hydrothermal shrinkage temperature of from 50° C. to 70° C.

In another aspect, this disclosure provides a method of preparing the purified insoluble type

II collagen fibers of any one of the preceding claims. In some embodiments, the method comprises: (a) obtaining type II collagen-rich articular cartilage tissues; (b) removing articular cartilage tissue from the cartilage tissue; (c) mechanically breaking down or cryo-milling the articular cartilage tissue; (d) performing freeze and thaw of the articular cartilage tissue for a minimum of three times; (e) treating the articular cartilage tissue of step (d) at a temperature≤4° C. with the following chemicals in any order: 0.05M to 0.5 M NH₄Cl, 0.05 M to 0.5 M NaOH, 0.05 M to 0.5 M HCl, 0.05 M to 0.5 M EDTA, 0.1 M to 4 M; and guanidine hydrochloride, to obtain the chemically treated and purified type II collagen fibers; and (f) washing the chemically treated and purified type II collagen fibers with H₂O.

In some embodiments, the method further comprises determining purity of the chemically treated and purified type II collagen fibers. In some embodiments, the method further comprises repeating steps (d)-(f).

In some embodiments, the type II collagen-rich articular cartilage tissues are knee joint articular cartilage. In some embodiments, the type II collagen-rich articular cartilage tissues are chicken sternum.

### DETAILED DESCRIPTION OF THE INVENTION

Type II collagen is an integral part of the articular cartilage extracellular matrix (ECM). In combination with proteoglycans, they form a physically, mechanically, and biochemically stable structure that provides the necessary properties for function in the joints. Due to the unique structure of the ECM, low cell counts in the tissue and lack of blood supply to nourish the cells, once injured from sports or disease, the tissue lacks the intrinsic capability to repair and regenerate the diseased and injured tissue. More often than not, with time, the injured tissue induces a chronic inflammatory response, which leads to loss of tissue and further tissue degeneration. Eventually, the disease would advance to the state of complete loss of cartilage tissue, and the opposite bones come in contact that require surgical intervention to replace the knee, i.e., the total knee replacement surgery.

To develop a type II collagen-based extracellular matrix (ECM) that is as close to the native ECM of the articular cartilage as possible, this disclosure describes highly purified insoluble type II collagen fibers that have a type II collagen content greater than 97% by weight with only residual amounts of non-collagenous materials and methods of obtaining same. This purified insoluble collagen matrix can advance science and technology to the next level for tissue engineering type II collagen fiber-based ECM for the treatment of articular cartilage lesions.

One objective of this invention is to provide an engineered ECM of the articular cartilage tissue that mimics the ECM in the natural tissue to support and guide the repair of the missing tissue and also to induce regeneration of articular cartilage for the return of normal function of the joint, e.g., the knee. To that end, one has to develop the key material, type II collagen fibers, from the healthy native cartilage tissue of the animals. One of the key hurdles at present is the isolation and purification of the intact type II collagen fibers for engineering ECM of the articular cartilage.

Currently, there are two types of materials which have been tried to engineer the tissue ECM matrices, namely synthetic polymeric materials, and biological-based materials. On the synthetic side, polymers like polyglycolic acid (PGA), polylactic acid (PLA), various PGA-PLA copolymers, and polycaprolactone (PCL) are among the popular materials. It is well recognized that synthetic materials are being responded to in vivo as foreign materials which can cause unwanted foreign body reactions from the host. The degradable products may also induce various extents of unwanted side reactions from some synthetic polymers. To overcome these disadvantages, some researchers incorporate biological materials, such as type I collagen-based hydrogel (containing about 10 mg/ml), type II collagen hydrogel (≤10 mg/ml), fibrin glue, alginate, and hyaluronan with the synthetic polymers in the form of a coating or via mixing of the two materials. Overall, pure synthetic materials or composite materials have not yet been able to engineer a scaffold that is suitable for treating articular cartilage injuries.

On the biological side, type I collagen fibers, type I collagen molecules (atelocollagen I), type II collagen molecules (atelocollagen II), hyaluronan, agarose, and alginate have been evaluated for engineering biocompatible 3D scaffold for articular cartilage tissue repair and regeneration. It is known that biological materials exhibited more cytocompatibility than synthetic polymers. As type II collagen is the critical component of the ECM of articular cartilage, type II collagen is considered to be the best candidate for engineering ECM scaffolds for cartilage repair. It has been demonstrated in in vitro cell culture studies; type II collagen materials are more suitable for engineering ECM scaffolds for articular cartilage tissue engineering applications.

In the category of type II collagen, soluble type II collagen molecules have been extracted from cartilage tissues in aqueous solutions in the presence of enzyme pepsin to cleave the intermolecular crosslinks and release collagen molecules into acidic solutions. However, the quantity of type II collagen molecules isolated is small. It can be used for research purposes but is not suitable for the development of implantable ECM scaffolds, not to mention the scale of clinical applications.

One of the inventors developed many types I collagen fiber-based ECM scaffold implants for guiding various tissue regeneration applications. However, type II collagen has a property different from type I collagen. First, type II collagen has a much smaller fibril size (nm range) than type I collagen (μm range). Type II collagen fibrils do not aggregate significantly while type I collagen fibrils aggregate into macroscopic fiber such as in tendon and ligament tissues.

Next, the intermolecular crosslinks in type I and type II collagens are distinctly different. Type I collagen contains mostly the crosslinks derived from the interaction of aldehyde group containing lysine (allysine) from the telopeptide of one molecule with the adjacent amino group of another lysine from the triple helical part in the adjacent molecule forming an acid labile double bond (Schiff base) which is later reduced to a stable single bond between two collagen molecules (divalent) in vivo. The crosslinks in articular cartilage are mostly in the stable form of hydroxylysyl pyridinolines involving three molecules (trivalent crosslinking).

In a type I collagen molecule, there are about an equal number of acidic groups derived from the carboxylic groups of aspartic and glutamic acids and basic groups derived from lysine and arginine side chains. Therefore, the fibers will swell in either acidic or basic solutions due to their divalent crosslinking structure. This is the key advantage of using type I collagen for tissue engineering applications to develop type I collagen-based scaffold implants. On the other hand, the stable trivalent crosslinks of pyridinoline-based intrafibrillar and interfibrillar crosslinks prevented swelling of the type II collagen fibrils and fibers which results in insensitivity of the type II collagen fibrils to acid or base treatment. Therefore, the trivalent intrafibril and interfibril crosslinking structure combining with the complex formation with the proteoglycan molecules result in the ECM of the articular cartilage difficult for type II collagen fiber isolation and purification. Various attempts to isolate and purify the insoluble type II collagen fibers have resulted in a solid type II collagen-based material partially degraded having a significant reduction of hydrothermal stability as compared to the intact tissue.

This disclosure provides purified insoluble type II collagen fibers. In some embodiments, the purified insoluble type II collagen fibers comprise less than 0.6% (w/w) (e.g., 0.6% (w/w), 0.55% (w/w), 0.5% (w/w), 0.45% (w/w), 0.4% (w/w), 0.35% (w/w), 0.3% (w/w), 0.25% (w/w), 0.2% (w/w), 0.15% (w/w), 0.1% (w/w), 0.05% (w/w), or any intermediate values therebetween) uronic acid.

In some embodiments, the type II collagen fibers comprise less than 0.3% (w/w) of uronic acid. In some embodiments, the type II collagen fibers comprise less than 0.1% (w/w) of uronic acid.

In some embodiments, the purified insoluble type II collagen fibers comprise less than 50 ng/mg (e.g., 50 ng/mg, 49 ng/mg, 48 ng/mg, 47 ng/mg, 46 ng/mg, 45 ng/mg, 44 ng/mg, 43 ng/mg, 42 ng/mg, 41 ng/mg, 40 ng/mg, 39 ng/mg, 38 ng/mg, 37 ng/mg, 36 ng/mg, 35 ng/mg, 34 ng/mg, 33 ng/mg, 32 ng/mg, 31 ng/mg, 30 ng/mg, 29 ng/mg, 28 ng/mg, 27 ng/mg, 26 ng/mg, 25 ng/mg, 24 ng/mg, 23 ng/mg, 22 ng/mg, 21 ng/mg, 20 ng/mg, 19 ng/mg, 18 ng/mg, 17 ng/mg, 16 ng/mg, 15 ng/mg, 14 ng/mg, 13 ng/mg, 12 ng/mg, 11 ng/mg, 10 ng/mg, 9 ng/mg, 8 ng/mg, 7 ng/mg, 6 ng/mg, 5 ng/mg, 4 ng/mg, 3 ng/mg, 2 ng/mg, 1 ng/mg, or any intermediate values therebetween) of nucleic acid molecules.

In some embodiments, the type II collagen fibers comprise less than 30 ng/mg of the nucleic acid molecules. In some embodiments, the type II collagen fibers comprise less than 10 ng/mg of the nucleic acid molecules. In some embodiments, the nucleic acid molecules comprise DNA.

In some embodiments, the purified insoluble type II collagen fibers comprise more than 12.5% (w/w) (e.g., 12.5% (w/w), 13% (w/w), 13.5% (w/w), 14% (w/w), 14.5% (w/w), 15% (w/w), 15.5% (w/w), or any intermediate values therebetween) of hydroxyproline.

In some embodiments, the type II collagen fibers comprise more than 13% (w/w) of the hydroxyproline. In some embodiments, the type II collagen fibers comprise more than 14% (w/w) of the hydroxyproline. In some embodiments, the type II collagen fibers comprise more than 15% (w/w) of the hydroxyproline.

In some embodiments, the purified insoluble type II collagen fibers comprise more than 97% (w/w) (e.g., 97% (w/w), 97.1% (w/w), 97.2% (w/w), 97.3% (w/w), 97.4% (w/w), 97.5% (w/w), 97.6% (w/w), 97.7% (w/w), 97.8% (w/w), 97.9% (w/w), 98% (w/w), 98.1% (w/w), 98.2% (w/w), 98.3% (w/w), 98.4% (w/w), 98.5% (w/w), 98.6% (w/w), 98.7% (w/w), 98.8% (w/w), 98.9% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), 99.9% (w/w), or any intermediate values therebetween) of type II collagen.

In some embodiments, the purified insoluble type II collagen fibers comprise less than 3% (w/w) (e.g., 3% (w/w), 2.9% (w/w), 2.8% (w/w), 2.7% (w/w), 2.6% (w/w), 2.5% (w/w), 2.4% (w/w), 2.3% (w/w), 2.2% (w/w), 2.1% (w/w), 2% (w/w), 1.9% (w/w), 1.8% (w/w), 1.7% (w/w), 1.6% (w/w), 1.5% (w/w), 1.4% (w/w), 1.3% (w/w), 1.2% (w/w), 1.1% (w/w), 1% (w/w), 0.9% (w/w), 0.8% (w/w), 0.7% (w/w), 0.6% (w/w), 0.5% (w/w), 0.4% (w/w), 0.3% (w/w), 0.2% (w/w), 0.1% (w/w), or any intermediate values therebetween) of collagens other than type II collagen and glycosaminoglycans (GAGs).

In some embodiments, the type II collagen fibers comprise less than 1% (w/w) of collagens other than type II collagen and GAGs. In some embodiments, the type II collagen fibers comprise less than 0.5% (w/w) of collagens other than type II collagen and GAGs.

In some embodiments, the collagen fibers are isolated from cartilage tissues of animals or humans.

In some embodiments, the tissues are knee joint articular cartilages. In some embodiments, the tissues are chicken sternum.

In some embodiments, the collagen fibers are isolated by genetic engineering technologies.
the collagen fibers have stability as measured by a hydrothermal shrinkage temperature of from 50° C. to 70° C. (e.g., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., or any intermediate values therebetween).

In another aspect, this disclosure provides a method of preparing the purified insoluble type II collagen fibers of any one of the preceding claims. In some embodiments, the method comprises: (a) obtaining type II collagen-rich articular cartilage tissues; (b) removing articular cartilage tissue from the cartilage tissue; (c) mechanically breaking down or cryo-milling the articular cartilage tissue; (d) performing freeze and thaw of the articular cartilage tissue for a minimum of three times; (e) treating the articular cartilage tissue of step (d) at a temperature≤4° C. with the following chemicals in any order: 0.05M to 0.5 M NH₄Cl, 0.05 M to 0.5 M NaOH, 0.05 M to 0.5 M HCl, 0.05 M to 0.5 M EDTA, 0.1 M to 4 M; and guanidine hydrochloride, to obtain the chemically treated and purified type II collagen fibers; and (f) washing the chemically treated and purified type II collagen fibers with H₂O.

In some embodiments, the method further comprises determining purity of the chemically treated and purified type II collagen fibers. In some embodiments, the method further comprises repeating steps (d)-(f).

In some embodiments, the type II collagen-rich articular cartilage tissues are knee joint articular cartilage. In some embodiments, the type II collagen-rich articular cartilage tissues are chicken sternum.

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the phrases “nucleic acid,” “polynucleotide,” “oligonucleotide,” and “nucleic acid molecule” are used interchangeably to refer to a polymer of DNA and/or RNA, which can be single-stranded, double-stranded, or multi-stranded, synthesized or obtained (e.g., isolated and/or purified) from natural sources, which can contain natural, non-natural, and/or altered nucleotides, and which can contain natural, non-natural, and/or altered internucleotide linkages including, but not limited to phosphoroamidate linkages and/or phosphorothioate linkages instead of the phosphodiester found between the nucleotides of an unmodified oligonucleotide.

As used herein, “in vitro” refers to events that occur in an artificial environment, e.g., in a test tube or reaction vessel, in cell culture, etc., rather than within a multi-cellular organism.

As used herein, “in vivo” refers to events that occur within a multi-cellular organism, such as a non-human animal.

It is noted here that, as used in this specification and the appended claims, the singular forms “a,” “an,” and “the” include plural reference unless the context clearly dictates otherwise.

As used herein, “including,” “comprising,” “containing,” or “having” and variations thereof are meant to encompass the items listed thereafter and equivalents thereof as well as additional subject matter unless otherwise noted.

As used herein, the phrases “in one embodiment,” “in various embodiments,” “in some embodiments,” and the like do not necessarily refer to the same embodiment, but may unless the context dictates otherwise.

As used herein, the terms “and/or” or “/” means any one of the items, any combination of the items, or all of the items with which this term is associated.

As used herein, the term “substantially” does not exclude “completely,” e.g., a composition which is “substantially free” from Y may be completely free from Y. Where necessary, the word “substantially” may be omitted from the definition of the present disclosure.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As used herein, the term “each,” when used in reference to a collection of items, is intended to identify an individual item in the collection but does not necessarily refer to every item in the collection. Exceptions can occur if explicit disclosure or context clearly dictates otherwise.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

The use of any and all examples, or exemplary language (e.g., “such as”) provided herein, is intended merely to better illuminate the invention and does not pose a limitation on the scope of the present disclosure unless otherwise claimed. No language in the specification should be construed as indicating any non-claimed element as essential to the practice of the present disclosure.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present disclosure. Nothing herein is to be construed as an admission that the present disclosure is not entitled to antedate such publication by virtue of prior disclosure. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

This example describes in more detail the development of highly purified intact insoluble type II collagen-based fibers for the engineering of articular cartilage ECM matrix for the treatment of articular cartilage lesions. One of the objectives is to preserve the native structure of the type II collagen fibers from various treatments. It is extremely important to recognize that the knee articular cartilage is a very tightly packed tissue that has a low permeability to molecules. To improve the chemical penetration, cartilage tissue must first be reduced to fine thin strips or fine particles to increase the accessibility of the internal space of the tissue to chemicals. This can be accomplished by mincing the cartilage into thin strips using a meat slicer or other cutting mechanism on the frozen tissue. Fine strips of thickness between 0.5 mm and 1 mm noodle-like tissues can be produced. Further, prior to chemical treatment, finely minced articular cartilage tissue can be processed via a freeze and thaw step to open up the pores of the tissue for chemical penetration. The freeze and thaw step can be repeated several times to facilitate the chemical treatment steps described below.

Another method is by cryo-milling technique at the liquid nitrogen temperature to minimize the heat denaturation from the compacting forces acting on the tissue. Particle sizes less than 100 μm can be collected for this purpose.

Initially trypsin was used in combination with a strong oxidating agent (H₂O₂) to remove the GAGs in the tissue. However, it was found that trypsin had a negative effect on the structure of type II collagen. Even though it was effective in removing GAGs from the tissue, it also significantly reduced the thermal stability of the purified matrix. The purified type II collagen-based material was unstable in the presence of pepsin at room temperature. It is known that pepsin does not attack the triple helical portion of the molecule; this indicates that the trypsin-treated type II collagen matrix was partially denatured.

Therefore, the approach described herein did not use any proteomic enzymes (e.g., trypsin) and oxidating chemicals (e.g., H₂O₂) to remove the non-collagenous proteins that are part of the proteoglycan molecules as trypsin in combination with H₂O₂ had a negative effect on the structure of type II collagen.

Next, various chemicals have been evaluated, and it was found that in order not to denature the type II collagen fibrillar structure, lower concentrations of inorganic acid (e.g., HCl), inorganic base (NaOH) and guanidine hydrochloride at low temperature (e.g., 4° C.), organic based salt such as NH₄Cl, are important chemicals to remove the non-collagenous materials from the complex tissues like articular cartilage.

Thus, cryo-milled tissue particles or finely minced noodle strips were subjected to low concentration of NaOH, HCL, NH₄Cl, and EDTA along with guanidine hydrochloride treatment for a defined period of time. Importantly, in acid and base treatments, the concentration is not to exceed 0.5 M at a temperature of 4° C. For guanidine hydrochloride treatment, the concentration should not exceed 4M. Guanidine hydrochloride treatment must be conducted at 4° C. or below to avoid any potential type II collagen denaturation. It is recommended that multiple runs of a defined procedure may be needed to reach the goal of purity as described for the disclosed type II collagen fibers.

### A Example Purification Procedure is Provided as Follows

Finely minced noodle-like tissue strips were frozen at −80° C. for one hour. Then the frozen tissue was thawed at room temperature for 1 h and washed in deionized water for 30 min. This step was repeated twice for a total of 3 times of freeze-thaw procedure. The following procedure was then performed at 4° C. unless otherwise stated.

10 g of freeze-thawed tissue was placed in a beaker containing 1000 ml of 0.2M NH₄Cl, pH7 solution and kept the solution under constant stirring for 18 hours. The solution was removed and replaced with a new 1000 ml of precooled 0.2 M NH₄Cl (pH 7) solution and kept the solution under constant stirring for 24 hrs.

The second NH₄Cl (pH 7) solution was removed and replaced with 1000 ml precooled 0.5 M NaOH solution, and the beaker was kept under constant stirring for 4 hrs. The NaOH was removed and replaced with a new 1000 ml precooled 0.5 M NaOH solution. The new NaOH solution was under constant stirring for 16 hours.

The pH of NaOH solution was adjusted to 7 by adding 3M HCl and kept stirring for 30 min, followed by washing 2 times with 300 ml of 1% NaCl, 30 min each and then with 300 ml water for 30 min.

Next, 500 ml of 0.5M HCl was added to the beaker and kept stirring the solution for 24 hours. followed by adjusting the pH of HCl to 7 by adding 3M NaOH and kept stirring for 30 min., followed by washing 2 times with 300 ml 1% NaCl, 30 min each, followed by washing with 300 ml of water 3 times each for 30 min.

Next, 500 ml 4% EDTA at pH 7.5 was added to the beaker for 20 hours under constant stirring followed by washing with 300 ml 1% NaCl each for 3 times, followed by washing with 300 ml water 3 times and adjusting pH to 7 with NaOH or HCl if needed.

Finally, the tissue was treated with 300 ml of 4M guanidine-HCl under constant stirring for 24 hours. After 24 hours, tissue was washed with 500 ml of water 3 times for 60 min each followed by lyophilization.

### Purity Testing of the Purified Type II Collagen Fibers

The following testing program was applied to quantify the purity of the purified type II collagen fibers. The tests involved in determining the purity of the type II collagen fibers are multifold. Each test was developed with a justification stated below.

### Uronic Acid Determination

Uronic acid accounts for about 50% of the total GAGs in the tissue. It would be feasible to remove most if not all of the uronic acid from the tissue as a very small quantity of GAGs are believed to be covalently associated with type II collagen. An upper limit of uronic acid in the final purified type II collagen fiber matrix was set to 0.6%.

### Method of Uronic Acid Determination:

The method of uronic acid determination was developed based on Bitter, T. and Muir, H. (Bitter, T and Muir, H M, 1962, A modified uronic acid carbazole reaction, Anal. Biochem. 4:330-334). Glucuronic acid was used as the standard. In this test, the solutions of the standard and the test samples were properly adjusted to minimize the background interference of the test.

### Sodium Dodecyl Sulfate Polyacrylamide Gel Electrophoresis (SDS-PAGE) Analysis

Purified type II collagen fibers were subjected to pepsin digestion to release type II collagen molecules from the insoluble matrix. SDS-PAGE analysis should only show one major band of al chain of the type II collagen. Any additional band above or below the al band would likely be due to the impurities of other collagen chains or partially degraded a chain of type II collagen.

### Method of SDS-PAGE Analysis:

SDS-PAGE analysis was performed using Mini-PROTEIN Tera Cell (Cat #1658004, Bio-Rad System) and precast gel and a PowerPac Basic Supply (Cat #1645050, Bio-Rad).

### Hydroxyproline Content

Hydroxyproline is a unique amino acid of collagen. In some embodiments, the hydroxyproline content of fiber-forming collagen such as type I, type II, and type III collagens is in the range of 13-14% by weight. A determination of the hydroxyproline content would indicate whether the material is pure collagen or contains other non-collagenous materials.

Method of hydroxyproline determination:

Hydroxyproline content was determined based on Bergman, I and Loxley, R (Analytical Chemistry, 12:1961-1965, 1963)

### DNA Content

DNA content determination of the purified type II collagen fibers would determine whether the remaining cell-based materials were removed during the purification process. In general, xenogeneic DNAs are immunogenic. If implants developed from animal tissues, the FDA had limited the DNA content to be <50 ng/mg of the implant to minimize the potential immune response from the implant.

### Method of DNA Determination:

### Method of DNA Content Determination:

DNA content was conducted according to DNeasy Blood & Tissue kit instruction manual from Qiagen (Cat #69504) and General DNA Quantification Kit instruction manual from Abcam (Cat #ab156902).

### Hydrothermal Shrinkage Temperature

Hydrothermal shrinkage temperature (Tₛ) provides information regarding the intactness of the type II collagen after extensive treatment from the various chemicals during the purification process. The Tₛ was determined via a differential scanning calorimetry (Mettler, Swiss)) under a control operating condition. Native tissue containing intact type II collagen fibers was used as a control for the intact native structure of collagen fibers. It is important that the operation conditions were defined for comparison purposes which include the heating rate (temperature increase per minute), the sample size (mg), the sample geometry in the sealed pan, and the hydration condition. All else was fixed, a heating rate of 5° C./min was used for every sample tested.

### Results of Testing of the Purified Insoluble Type II Collagen Preparation

The table below summarizes the testing results of the purified insoluble type II collagen fibers prepared from the current invention.

| | |
|---|---|
| Uronic Acid (%) | <0.6 |
| SDS-PAGE (% type II collagen based on density of the band | >97 |
| analysis) | |
| Hydroxyproline content (%) | >12.5 |
| DNA content (ng/mg) | <50 |
| Hydrothermal stability (° C. at 5° C./min speed) | >50 |

The test results demonstrated that the purified insoluble type II collagen-based fiber matrix contains at least 97% by weight of type II collagen and less than 3% of other types of collagens and the residual number of GAGs. The DNA content is below the safety level. The purified insoluble type II collagen fiber material can be used for medical implant development applications.

The present disclosure is not to be limited in scope by the specific embodiments described herein. Indeed, various modifications of the invention, in addition to those described herein, will become apparent to those skilled in the art from the foregoing description and the accompanying figures. Such modifications are intended to fall within the scope of the appended claims.

What is claimed is:

1. Purified insoluble type II collagen fibers, comprising:
less than 0.6% (w/w) uronic acid,
less than 50 ng/mg of nucleic acid molecules,
from 12.5% (w/w) to 15.5% (w/w) of hydroxyproline,
more than 97% (w/w) of type II collagen, and
less than 3% (w/w) of collagens other than type II collagen and glycosaminoglycans (GAGs).

2. The type II collagen fibers of claim 1, comprising less than 0.3% (w/w) of the uronic acid.

3. The type II collagen fibers of claim 1, comprising less than 0.1% (w/w) of the uronic acid.

4. The type II collagen fibers of claim 1, comprising less than 30 ng/mg of the nucleic acid molecules.

5. The type II collagen fibers of claim 1, comprising less than 10 ng/mg of the nucleic acid molecules.

6. The type II collagen fibers of claim 1, wherein the nucleic acid molecules comprise DNA.

7. The type II collagen fibers of claim 1, comprising more than 13% (w/w) of the hydroxyproline.

8. The type II collagen fibers of claim 1, comprising more than 14% (w/w) of the hydroxyproline.

9. The type II collagen fibers of claim 1, comprising more than 15% (w/w) of the hydroxyproline.

10. The type II collagen fibers of claim 1, comprising less than 1% (w/w) of collagens other than type II collagen and GAGs.

11. The type II collagen fibers of claim 1, comprising less than 0.5% (w/w) of collagens other than type II collagen and GAGs.

12. The type II collagen fibers of claim 1, wherein the collagen fibers are isolated from cartilage tissues of animals or humans.

13. The type II collagen fibers of claim 12, wherein the tissues are knee joint articular cartilages.

14. The type II collagen fibers of claim 12, wherein the tissues are chicken sternum.

15. The type II collagen fibers of claim 1, wherein the collagen fibers are isolated by genetic engineering technologies.

16. The type II collagen fibers of claim 1, wherein the collagen fibers have stability as measured by a hydrothermal shrinkage temperature of from 50° C. to 70° C.

17. A method of preparing the purified insoluble type II collagen fibers of any one of the preceding claims, comprising:
(a) obtaining type II collagen-rich articular cartilage tissues;
(b) removing articular cartilage tissue from the cartilage tissue;
(c) mechanically breaking down or cryo-milling the articular cartilage tissue;
(d) performing freeze and thaw of the articular cartilage tissue for a minimum of three times;
(e) treating the articular cartilage tissue of step (d) at a temperature≤4° C. with the following chemicals in any order: 0.05M to 0.5M NH₄Cl, 0.05M to 0.5M NaOH, 0.05M to 0.5M HCl, 0.05M to 0.5M EDTA, 0.1M to 4M; and guanidine hydrochloride, to obtain the chemically treated and purified type II collagen fibers; and
(f) washing the chemically treated and purified type II collagen fibers with H₂O.

18. The method of claim 17, further comprising determining purity of the chemically treated and purified type II collagen fibers.

19. The method of claim 17, comprising repeating steps (d)-(f).

20. The cartilage tissues of claim 17, wherein the type II collagen-rich articular cartilage tissues are knee joint articular cartilage.

21. The cartilage tissues of claim 17, wherein the type II collagen-rich articular cartilage tissues are chicken sternum.
